(19) United States
(12) Patent Application Publication
(10) Pub. No.: US 2012/0309989 A1
(43) Pub. Date: Dec. 6, 2012

(54) CONTINUOUS PROCESS FOR THE PRODUCTION OF BETA-KETO ESTERS BY CLAISEN CONDENSATION

(75) Inventors: Peter McCormack, Wirral Merseyside (GB); Antony John Warr, Wirral Merseyside (GB); Elliot Latham, Wirral Merseyside (GB)

(73) Assignee: PHOENIX CHEMICALS LIMITED, Wirral Merseyside (GB)

(21) Appl. No.: 13/522,455

(22) PCT Filed: Oct. 22, 2010
(86) PCT No.: PCT/GB10/51778

§ 371 Date: Jul. 16, 2012

(30) Foreign Application Priority Data
Oct. 23, 2009 (GB) 0918613.1

(51) Int. Cl. C07C 253/18; C12P 13/00; C07D 319/06; C07C 255/17

(52) U.S. Cl. 549/373; 558/440; 435/128

(57) ABSTRACT

The invention concerns A continuous process for the production of compounds having the general formula (6):

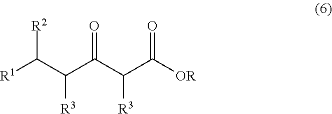

wherein R is a straight or branched chain alkyl group, R¹ is a straight or branched chain alkyl group substituted with a nitrile group, a hydroxy group or a halogen atom, R² is a hydroxy group or a keto group, and each R³ is, independently, hydrogen or a straight or branched chain alkyl group, comprising providing to a reaction zone a continuous stream of an alkyl acetate and a continuous stream of an alkali metal or alkaline earth metal amide base and contacting the continuous streams together in the reaction zone to yield an enolate compound, providing to the or a separate reaction zone a continuous stream of a compound of formula (5):

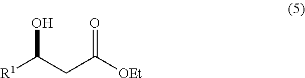

wherein R¹ is as previously defined, and contacting the continuous stream of compound (5) with a continuous stream of the enolate (4) in the or the separate reaction zone at a temperature above 20° C. to yield an intermediate compound and treating the intermediate compound of formula (1) with an acid to yield the compound of formula (6).

The present invention concerns a process for the production of certain pharmaceutically useful intermediate compounds, in particular (5R)-1,1-dimethylethyl-6-cyano-5-hydroxy-3-oxo-hexanoate.

(5R)-1,1-dimethylethyl-6-cyano-5-hydroxy-3-oxo-hexanoate is a useful pharmaceutical intermediate particularly in the manufacture of statin drugs such as atorvastatin, sold under the trade name Lipitor™

(5R)-1,1-dimethylethyl-6-cyano-5-hydroxy-3-oxo-hexanoate is conventionally manufactured batchwise by a Claisen type reaction between tertiary butyl acetate (strictly speaking, the enolate of tertiary butyl acetate) and (3R)-4-cyano-3-hydroxybutyric acid, ethyl ester. However, the enolate is unstable above −30° C. At 0-5° C. in THF at concentrations of about 1.5M the compound begins to decompose in less than 1 minute and is substantially decomposed in around 5 mins.

Decomposition of the enolate can take place in accordance with the following scheme:

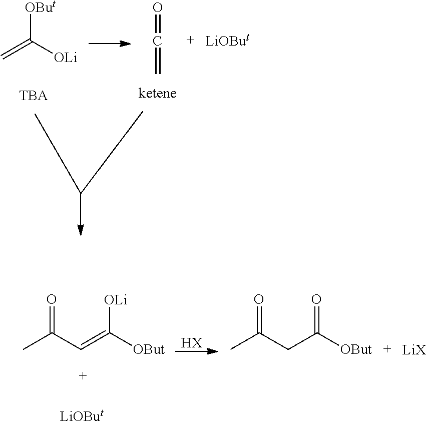

Tertiary butyl acetate enolate (TBA in the above scheme) decomposes to the ketene which then reacts with another molecule of tert-butyl acetate enolate to self condense to give tert-butylacetoacetate. tert-Butylacetoacetate is the major impurity in all Claisen type reactions involving TBA. Since the reagents that go to make tert-butyl acetate enolate, in particular the lithium amide base, are expensive, the formation of tert-butylacetoacetate is a costly inefficiency. As batching operations/heat transfer takes hours not minutes at industrial scale it is thus necessary in order to prepare and use the enolate to utilise reactor temperatures as low as −30° C. or lower (as taught in EP-A-0643689). Thus, conventionally, cryogenic reactors are required for good reagent efficiency and yield to be obtained.

It has been suggested, for example in U.S. Pat. No. 6,903,225 and in U.S. Pat. No. 6,340,767, to use higher temperatures, but these disclosures appear to address the problem of enolate self-condensation by forming the enolate very slowly by dropwise addition of base to acetate over a three hour period and by carrying out the enolate formation reaction in the presence of the other reaction partner 4-chloro-3-hydroxybutyric acid ethyl ester, conditions which are seemingly unlikely to be commercially attractive on an industrial scale. Another apparent disadvantage of this process is that in some cases it may not be possible to carry out the enolisation reaction in the presence of the other reaction partner, particularly if this reaction partner is sensitive to the strong bases such as lithium amides that are used for such enolisations.

It is an object of the present invention to address these problems.

According to the present invention there is provided a continuous process for the production of compounds having the general formula (6):

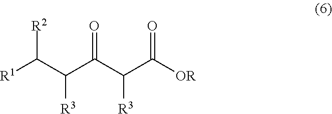

wherein:
   R is a straight or branched chain alkyl group;
   R¹ is a straight or branched chain alkyl group substituted with a nitrile group, a hydroxy group or a halogen atom;
   R² is a hydroxy group or a keto group; and
   each R³ is, independently, hydrogen or a straight or branched chain alkyl group,
the process comprising providing to a reaction zone a continuous stream of a compound of formula (3):

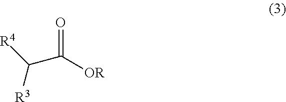

wherein R and R³ are as previously defined and R⁴ is hydrogen or has the general formula (7):

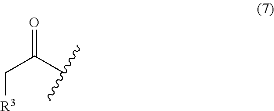

wherein R³ is as defined above and a continuous stream of an alkali metal or alkaline earth metal amide base, alkyl lithium or Grignard reagent; contacting the continuous streams together in the reaction zone to yield the enolate of formula (4):

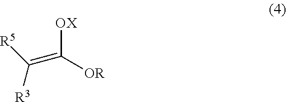

wherein R and R³ are as previously defined, X is an alkali metal or alkaline earth metal, and R⁵ is hydrogen or has the general formula (8):

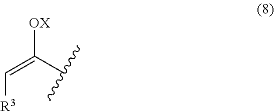

wherein R³ and X are as previously defined;
providing to the or a separate reaction zone a continuous stream of a compound of formula (5):

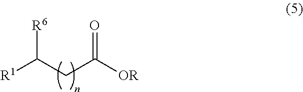

wherein R and R¹ are as previously defined or together define a ring structure, R⁶ is hydrogen, hydroxyl, alkoxyl or a keto group and n is 0 or 1;
and contacting the continuous stream of compound (5) with a continuous stream of the enolate (4) in the or the separate reaction zone at a temperature above 20° C. to yield a compound of formula (I):

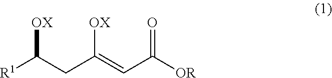

wherein R¹, R and X are as previously defined, and treating the compound of formula (I) with an acid to yield the compound of formula (6).

We have found that by conducting this Claisen type reaction under continuous conditions, it is possible to operate the process at significantly higher temperatures than have hitherto been considered workable, whilst obtaining good yields and purity. As a consequence, the process of the invention does not require cryogenic cooling equipment, and provides the compound (6) product in good yields and purities, notwithstanding the relatively high temperature of operation.

Conventionally, enolates of the type represented by formula (4) are prepared at low temperature due to their thermal instability. For similar reasons, the reaction between compound (4) and compound (5) also conventionally takes place at low temperature. This is because on an industrial scale if one prepares an 8000 L batch of enolate mixture at −60° C. and one wants to carry out a subsequent reaction at 10° C. using this enolate solution it is not possible to warm this solution to 10° C. at a rate faster than the enolate will decompose.

Lying behind the present invention is the realisation that it is possible prepare the enolate at a higher than conventional temperature and use it immediately also at a higher than conventional temperature. In the context of a continuous process which allows the operator very rapidly (preferably over a time frame of minutes or seconds) to mix the enolate with a reaction partner (compound (5)). In the continuous process of the invention the enolate is enabled to react with its reaction partner (for example 4-cyano-3-hydroxybutyric acid ethyl ester) at a faster rate than it would decompose (self-condense). A significant advantage of the inventive process is therefore that it allows the use of non-cryogenic conditions in both the synthesis and use of ester enolates, and also for rapid production of compound (6) on an industrial scale.

Preferably in the process of the invention, the enolisation reaction is conducted at least partially before contacting the enol compound (4) with its reaction partner compound (5). In other words, preferably the steps of providing to the reaction zone a continuous stream of a compound of formula (3) and a continuous stream of an alkali metal or alkaline earth metal amide base, alkyl lithium or a Grignard reagent, and the steps of providing to the or the separate reaction zone a continuous stream of a compound of formula (5) are sequential steps in the process of the invention. This aspect of the invention is found to be particularly advantageous when compound (5) is itself unstable in the presence of the alkali metal or alkaline earth metal amide base, alkyl lithium or a Grignard reagent, as appears to be the case for example when R¹ contains a nitrile group.

We have found that under the continuous operating conditions of the process of the invention it is also possible to reduce the stoichiometric ratio of alkali metal or alkaline earth metal amide base, alkyl lithium or a Grignard reagent to compound of formula (5) below the level conventionally employed, an important advantage given the expense and/or difficulty of manufacturing the alkali metal or alkaline earth metal amide base, alkyl lithium or Grignard reagent.

Accordingly, in one preferred process according to the invention, the stoichiometric ratio of alkali metal or alkaline earth metal amide base, alkyl lithium or a Grignard reagent to compound (5) supplied to the and/or to the separate reaction zone is less than about 4.5:1, more preferably less than about 4.0:1 and most preferably less than about 3.5:1.

Continuous flow production of the unstable compound (4) allows the compound to be used as it is formed, and allows the use of very high heat/mass transfer flow equipment, permitting excellent temperature control of the reaction mixture.

Consequently, the synthesis and use of compound (4) may be effected in the process of the invention at higher temperatures than typically observed for batch type reactor systems.

Preferably the temperature at which compounds (4) and (5) are reacted together is above 25° C., more preferably above 30° C.

A significant advantage of using a relatively high temperature in the reaction between compounds (4) and (5) is that only a low residence time in the or the separate reaction zone need be employed.

Preferably the residence time of the contacted continuous streams of compounds (4) and (5) in the or the separate reaction zone is less than about 5 minutes, more preferably less than about 1 minute, still more preferably less than about 50 seconds and most preferably less than about 40, or even 30 seconds.

Preferably the residence time of the contacted continuous streams of compound (3) and the alkali metal or alkaline earth metal amide base, alkyl lithium or Grignard reagent in the reaction zone is less than about 5 minutes, more preferably less than about 4 minutes, still more preferably less than about 3 minutes and most preferably less than about 2 minutes.

Preferably the enolate compound (4) is prepared from the reaction of compound (3) and the alkali metal or alkaline earth metal amide base, alkyl lithium or Grignard reagent in a first reaction zone, and the compound of formula (I) is prepared from the reaction between compounds (4) and (5) in a second reaction zone. We have found using separate reaction zones to be preferable in terms of yield and/or purity, particularly when R¹ contains a nitrile group, compound (6) tending to have a red colouration due to impurities when the same reaction zone is used for both reactions. However, the use of first and second reaction zones is preferable rather than essential in the process of the invention and, particularly when R¹ contains a halogen atom, does not appear to compromise purity of the product unduly.

The treatment of compound (I) with acid may take place in the same or a different reaction zone as that in which the reaction between compounds (4) and (5) takes place, and this step of the process need not be continuous, although it can be.

By “continuous” is preferably meant that steady state reaction conditions prevail in the or the separate reaction zone as far as the reactions between compound (3) and the alkali metal or alkaline earth metal amide base, alkyl lithium or Grignard reagent and/or between compounds (4) and (5) are concerned. However reagent streams may be supplied to the or the separate reaction zone, and product stream(s) may be recovered therefrom as consistent continuous streams or as intermittent or pulsed streams.

R¹ is preferably a substituted methyl group. When R¹ is substituted with a halogen atom, the halogen atom is preferably chlorine.

R is preferably tertiary butyl.

X is preferably lithium and the alkali metal or alkaline earth metal amide base is preferably a lithium amide base, such as lithium hexamethyldisilazane or lithium diiospropylamide, lithium dicyclohexylamide or lithium amide.

Preferred processes in accordance with the invention for the preparation of particular compounds (6) in accordance with the process of the invention are summarised in the following Table 1 wherein each particularly preferred starting material (3) is shown in the first column, each alkali metal or alkaline earth metal amide base, alkyl lithium or Grignard reagent (indicated by the word “base”) is shown in the second column, each resulting enolate (4) is shown in the third column, each reaction partner (5) is shown in the fourth column and each target compound (6) is shown in the fifth column:

TABLE 1

| | | | | |
|---|---|---|---|---|
| (3) | (base) | (4) | (5) | (6) |

| | | | |
|---|---|---|---|
| 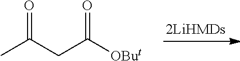 | 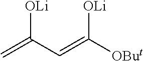 | 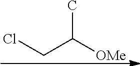 | 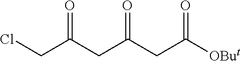 |
| 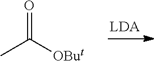 | 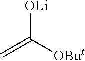 | 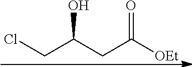 | 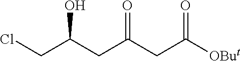 |
| 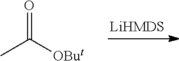 | 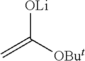 | 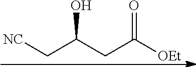 | 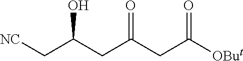 |
| 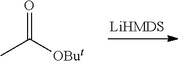 | 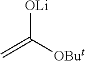 | 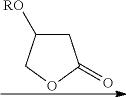 | 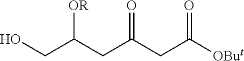 |

Where stereochemistry is specified in the table above, it should be understood that the process of the invention is also directed towards all stereoisomers and enantiomers.

Also provided in accordance with the present invention is a process for the preparation of compound (7):

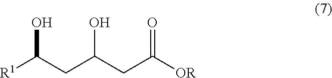

comprising obtaining compound (6) by the aforementioned process and subjecting that compound to reducing conditions to obtain compound (7). Preferably the reducing conditions are at least partially provided by one or more enzymes.

Also provided in accordance with the invention is a process for the preparation of compound (8):

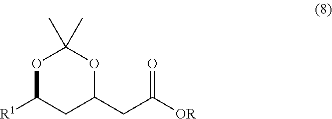

comprising obtaining compound (7) by the aforementioned process and subjecting that compound to acetalising conditions in the presence of an acid catalyst to obtain compound (8).

The invention also provides a process for the preparation of compound (8) as aforesaid and further converting compound (8) into a useful pharmaceutical compound.

The invention will now be more particularly described with reference to the following example.

### EXAMPLE

### Synthetic Sequence

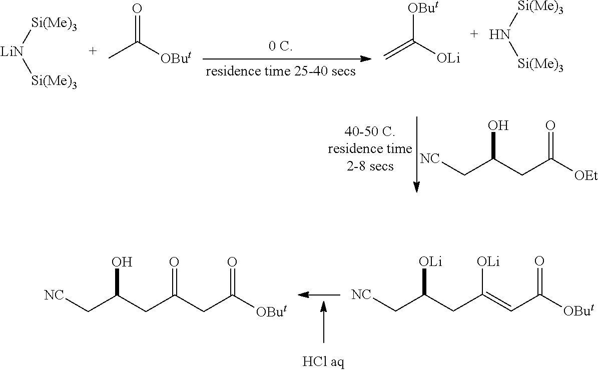

### Preparation of Tert-Butyl Acetate Enolate

t-Butyl Acetate enolate was prepared by pumping two solutions through a 1.016 mm i.d. stainless steel capillary tube:
1. A solution of lithium hexamethyldisilazane (24.36% w/w in THF) at a flow rate of 53.02 ml/min
2. A solution of tert-butyl acetate (50% w/w in THF) at a flow rate of 19.77 ml/min.

This gave very rapid and intimate mixing of the two solutions and a residence time for the reaction of 26.5 secs. The reaction temperature was controlled by submerging the entire capillary reactor in a Huber heater/chiller unit with a set-point of 0° C.

### Preparation of (R)-6-Cyano-5-hydroxy-3-oxo-hexanoic acid tert-butyl ester

The t-butyl acetate enolate stream was then immediately mixed with a flow of ethyl (R)-4-cyano-3-hydroxybutyrate (50% w/w in THF) (flow rate of 6.15 ml/min) and reacted in another stainless steel 1.016 mm i.d. capillary tube for a residence time of 2.4 secs. This gave very rapid and intimate mixing of the two solutions. The reaction temperature was controlled by submerging the entire capillary reactor in a water bath with a set-point of 55° C. The product stream was then cooled prior to quench by flowing through a 1.76 mm i.d. stainless steel capillary tube where the reaction temperature was controlled by submerging the reactor in an ice/water bath for a residence time of 3.6 secs.

### Reaction Quench/Work-Up

This mixture was then quenched into hydrochloric acid solution (1.7 Lts, 10% w/w) in a jacketed stirred glass reactor where the temperature was maintained at <25° C. using Huber heater/chiller unit. The pH was not allowed to rise above 2. Upon completion the agitator was stopped and the reaction mixture was allowed to separate, and the lower aqueous layer was split (3650 g), and extracted with dichloromethane (2×250 ml). The upper organic layer (3683 g) was combined with the organic extracts, and washed with water (2×250 ml). The organic extract was concentrated via a rotary film evaporator (bath temp 35° C.) to give crude (R)-6-Cyano-5-hydroxy-3-oxo-hexanoic acid tert-butyl ester (304.3 g) as a yellow/brown oil. The yield for the reaction was 72% as determined by ¹HNMR analysis using tridecane as internal standard. (¹HNMR analysis procedure: a 10 sec sample of unquenched product stream was added to a mixture of dichloromethane (5 ml), tridecane (450 μl) and hydrochloric acid (10% w/w, 5 ml). After briefly shaking, the mixture was allowed to separate and the lower organic layer was split and dried over magnesium sulphate. The sample was concentrated using a nitrogen sparge to give an oil, which was diluted with CDCl3 and analysed).

This example is intended only to illustrate the invention, which is more particularly defined in the claims which follow.

1. A continuous process for the production of compounds having the general formula (6): 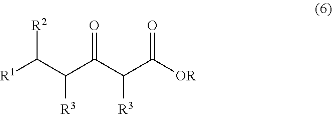
wherein:
R is a straight or branched chain alkyl group;
R¹ is a straight or branched chain alkyl group substituted with a nitrile group, a hydroxy group or a halogen atom;
R² is a hydroxy group or a keto group; and
each R³ is, independently, hydrogen or a straight or branched chain alkyl group,
the process comprising providing to a reaction zone a continuous stream of a compound of formula (3): 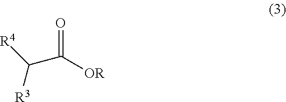
wherein R and R³ are as previously defined and R⁴ is hydrogen or has the general formula (7): 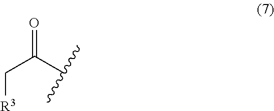
wherein R³ is as defined above
and a continuous stream of an alkali metal or alkaline earth metal amide base, alkyl lithium or Grignard reagent; contacting the continuous streams together in the reaction zone to yield the enolate of formula (4): 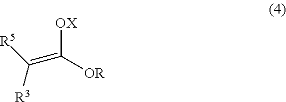
wherein R and R³ are as previously defined, X is an alkali metal or alkaline earth metal, and R⁵ is hydrogen or has the general formula (8): 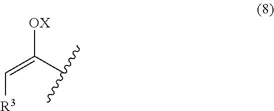
wherein R³ and X are as previously defined;
providing to the or a separate reaction zone a continuous stream of a compound of formula (5): 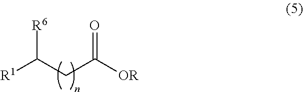
wherein R and R¹ are as previously defined or may together form a ring structure, R⁶ is hydrogen, hydroxyl, alkoxyl or a keto group and n is 0 or 1;
and contacting the continuous stream of compound (5) with a continuous stream of the enolate (4) in the or the separate reaction zone at a temperature above 20° C. to yield a compound of formula (I): 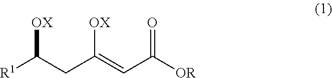
wherein R′, R and X are as previously defined, and treating the compound of formula (I) with an acid to yield the compound of formula (6).

2. A process according to claim 1 wherein the stoichiometric ratio of alkali metal or alkaline earth metal amide base, alkyl lithium or a Grignard reagent to compound (5) supplied to the and/or to the separate reaction zone is less than about 4.5:1.

3. A process according to claim 1 wherein the steps of providing to the reaction zone a continuous stream of a compound of formula (3) and a continuous stream of an alkali metal or alkaline earth metal amide base, alkyl lithium or Grignard reagent, and the steps of providing to the or the separate reaction zone a continuous stream of a compound of formula (5) are sequential steps.

4. A process according to claim 1 wherein the temperature at which compounds (4) and (5) are reacted together is above 25° C.

5. A process according to claim 4 wherein the temperature at which compounds (4) and (5) are reacted together is above 30° C.

6. A process according to claim 1 wherein the residence time of the contacted continuous streams of compounds (4) and (5) in the or the separate reaction zone is less than about 5 minutes.

7. A process according to claim 6 wherein the residence time of the contacted continuous streams of compounds (4) and (5) in the or the separate reaction zone is less than about 1 minute.

8. A process according to claim 7 wherein the residence time of the contacted continuous streams of compounds (4) and (5) in the or the separate reaction zone is less than about 50 seconds.

9. A process according to claim 8 wherein the residence time of the contacted continuous streams of compounds (4) and (5) in the or the separate reaction zone is less than about 40 seconds.

10. A process according to claim 1 wherein the residence time of the contacted continuous streams of compound (3) and the alkali metal or alkaline earth metal amide base, alkyl lithium or Grignard reagent in the reaction zone is less than about 5 minutes.

11. A process according to claim 1 wherein the enolate compound is prepared from the reaction of compound (3) and the alkali metal or alkaline earth metal amide base, alkyl lithium or Grignard reagent in a first reaction zone, and the compound of formula (I) is prepared from the reaction between compounds (4) and (5) in a second reaction zone.

12. A process according to claim 1 wherein steady state reaction conditions prevail in the or the separate reaction zone with respect to the reactions between compound (3) and the alkali metal or alkaline earth metal amide base, alkyl lithium or Grignard reagent and/or between compounds (4) and (5).

13. A process according to claim 1 wherein R¹ is a nitrile group or a chlorine atom.

14. A process according to claim 1 wherein R is tertiary butyl.

15. A process according to claim 1 wherein R¹ is a substituted methyl group.

16. A process according to claim 1 wherein X is lithium and the alkali metal or alkaline earth metal amide base is a lithium amide base.

17. A process for the preparation of compound (7): 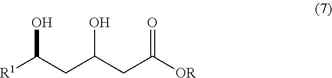
comprising obtaining compound (6) by the process of claim 1 and subjecting that compound to reducing conditions to obtain compound (7).

18. A process according to claim 17 wherein the reducing conditions are at least partially provided by one or more enzymes.

19. A process for the preparation of compound (8): 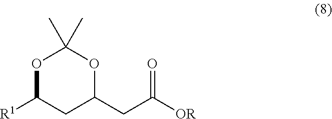
comprising obtaining compound (7) by the process of claim 17 and subjecting that compound to acetalising conditions in the presence of an acid catalyst to obtain compound (8).

20. A process for the preparation of compound (8) according to claim 19 and further converting compound (8) into a useful pharmaceutical compound.
